(19) United States
(12) Patent Application Publication
(10) Pub. No.: US 2015/0328040 A1
(43) Pub. Date: Nov. 19, 2015

(54) USER SUPPORT APPARATUS TO PROVIDE ENHANCED LIMB MOBILITY

(71) Applicant: Allan John Kloiber, JR., Great Falls, MT (US)

(72) Inventor: Allan John Kloiber, JR., Great Falls, MT (US)

(21) Appl. No.: 14/711,608

(22) Filed: May 13, 2015

Related U.S. Application Data
(60) Provisional application No. 61/992,283, filed on May 13, 2014.

(51) Int. Cl. A61F 5/37; A61G 5/12

(52) U.S. Cl. CPC A61F 5/3769; A61F 5/3792; A61G 5/12

(57) ABSTRACT

An apparatus to support a user's limb enhances the range of motion of the limb and permits the positioning of a counterforce to be adjusted, which corresponds to the weight of the limb. The apparatus includes a vertical member having a first end coupled to a structure and a second end having a spring mechanism, a horizontal arm assembly rotatably mounted to the spring mechanism by a joint assembly, the horizontal arm assembly having a first arm member coupled to a first side of the joint assembly and a second arm member coupled to a second side of the joint assembly, a limb support assembly coupled to the first arm member of the horizontal arm assembly and having a cuff to enclose a portion of the limb, and a counterweight member slidably mounted to the second arm member.

### RELATED APPLICATION

The application claims priority to provisional patent application U.S. Ser. No. 61/992,283 filed on May 13, 2014, the entire contents of which is herein incorporated by reference.

### BACKGROUND

The embodiments herein relate generally to devices for supporting a portion of a user's body such as an arm or leg.

Adults or children who suffer from various ailments and/or diseases sometimes have weakened muscles. As a result, these individuals have a difficult time performing simple tasks with their arms and/or legs due to a lack of strength and/or coordination. Slings can be wrapped around a portion of the user's body to help support and stabilize an arm or wrist. However, these devices are limited in that they are bulky or awkward, and do not provide the user's limb with a free range of motion.

Several arm support devices are disclosed in U.S. Pat. No. 6,224,026 and U.S. Patent Application Publication 2013/0087154, which include assemblies comprising components secured to the user's arm and a balance mechanism. However, these devices are disadvantageous because they either limit the user's range of motion of an arm and/or comprise complex components such as a spring-loaded reel system that attaches to the arm, which is subject to wear, premature failure and costly repairs. In addition, these devices do not enable an operator to easily adjust the positioning of the counterbalancing force created by the assembly to efficiently support the user's arm.

As such, there is a need in the industry for a user support apparatus for use in supporting one or more limbs, which addresses the limitations of the prior art and enhances limb mobility.

### SUMMARY

A support apparatus for use with a user to support a limb of the user and permit an enhanced range of motion of the user's limb is provided. The support apparatus is configured to adjust a positioning of a counterforce that corresponds to a weight of the limb. The support apparatus comprises a generally vertical member comprising a first end coupled to a support structure and a second end comprising a spring mechanism coupled thereto, a generally horizontal arm assembly rotatably mounted to the spring mechanism by a joint assembly, the horizontal arm assembly comprising a first arm member coupled to a first side of the joint assembly and a second arm member coupled to a second side of the joint assembly, a limb support assembly coupled to the first arm member of the horizontal arm assembly and comprising a cuff to enclose a portion of the limb, and a counterweight member slidably mounted to the second arm member, wherein the counterweight member generates the counterforce corresponding to the weight of the limb, thereby permitting the cuff to fully support the user's limb.

In some embodiments of the invention, the support apparatus comprises a vertical arm assembly with a first vertical arm and a second vertical arm. Each vertical arm is connected to the horizontal arm assembly, limb support assembly and counterweight member. This permits the support apparatus to be used with a pair of the user's limbs simultaneously.

### BRIEF DESCRIPTION OF THE FIGURES

The detailed description of some embodiments of the invention will be made below with reference to the accompanying figures, wherein the figures disclose one or more embodiments of the present invention.

FIG. 1 depicts a perspective view of certain embodiments of the support apparatus shown in use;

FIG. 2 depicts a perspective view of certain embodiments of the support apparatus;

FIG. 3 depicts an exploded view of certain embodiments of the support apparatus;

FIG. 4 depicts a reverse perspective view of ball coupling post 20 and socket sleeve 26 used in certain embodiments of the support apparatus;

FIG. 5 depicts an exploded view of line mount assembly 48 used in certain embodiments of the support apparatus;

FIG. 6 depicts a section view of certain embodiments of the support apparatus taken along line 6-6 in FIG. 2;

FIG. 7 depicts a section view of certain embodiments of the support apparatus;

FIG. 8 depicts an exploded view of counterweight 76, knob 42 and knob post 44 used in certain embodiments of the support apparatus;

FIG. 9 depicts an exploded view of a clamp assembly used in certain embodiments of the support apparatus;

FIG. 10 depicts a perspective view of an alternative clamp assembly used in certain embodiments of the support apparatus;

FIG. 11 depicts a perspective view of Y-riser assembly 108 used in certain embodiments of the support apparatus;

FIG. 12 depicts a perspective view of alternative base plate 116 used in certain embodiments of the support apparatus;

FIG. 13 depicts a rear perspective view of chair mount assembly 120 used in certain embodiments of the support apparatus;

FIG. 14 depicts a perspective view of certain embodiments of the support apparatus; and

FIG. 15 depicts a perspective view of certain embodiments of the support apparatus.

### DETAILED DESCRIPTION OF CERTAIN EMBODIMENTS

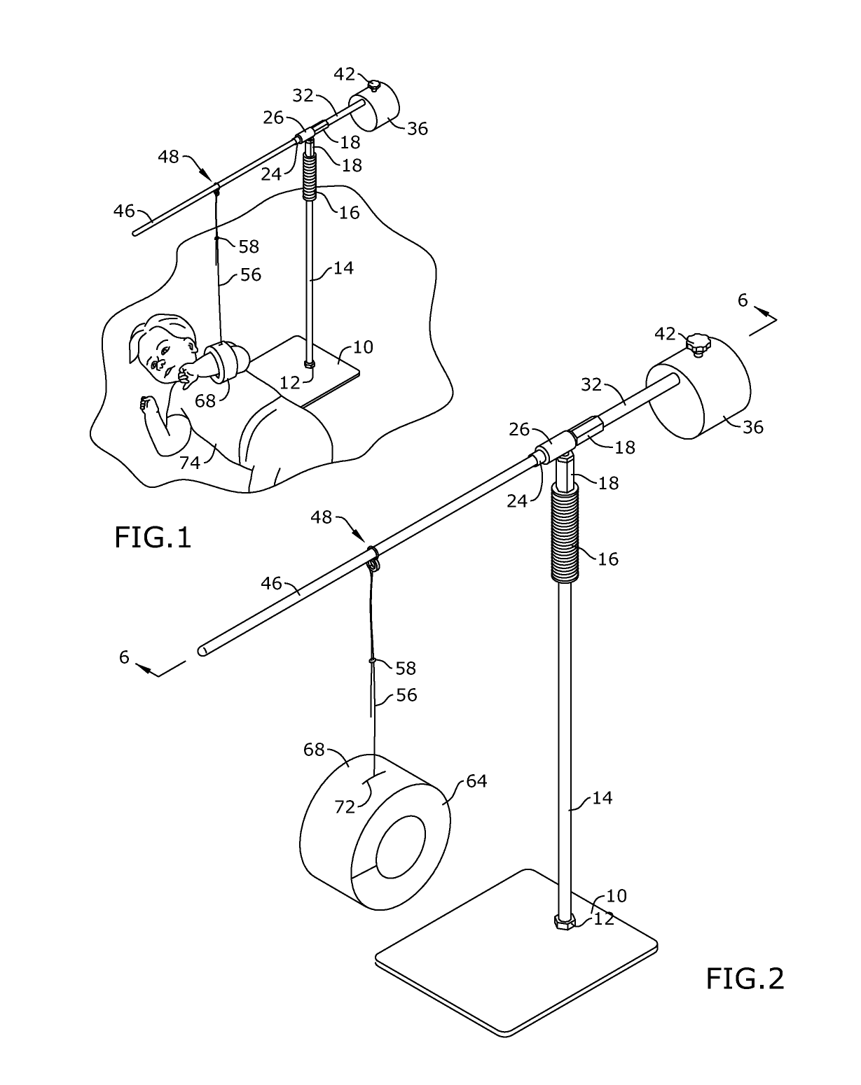

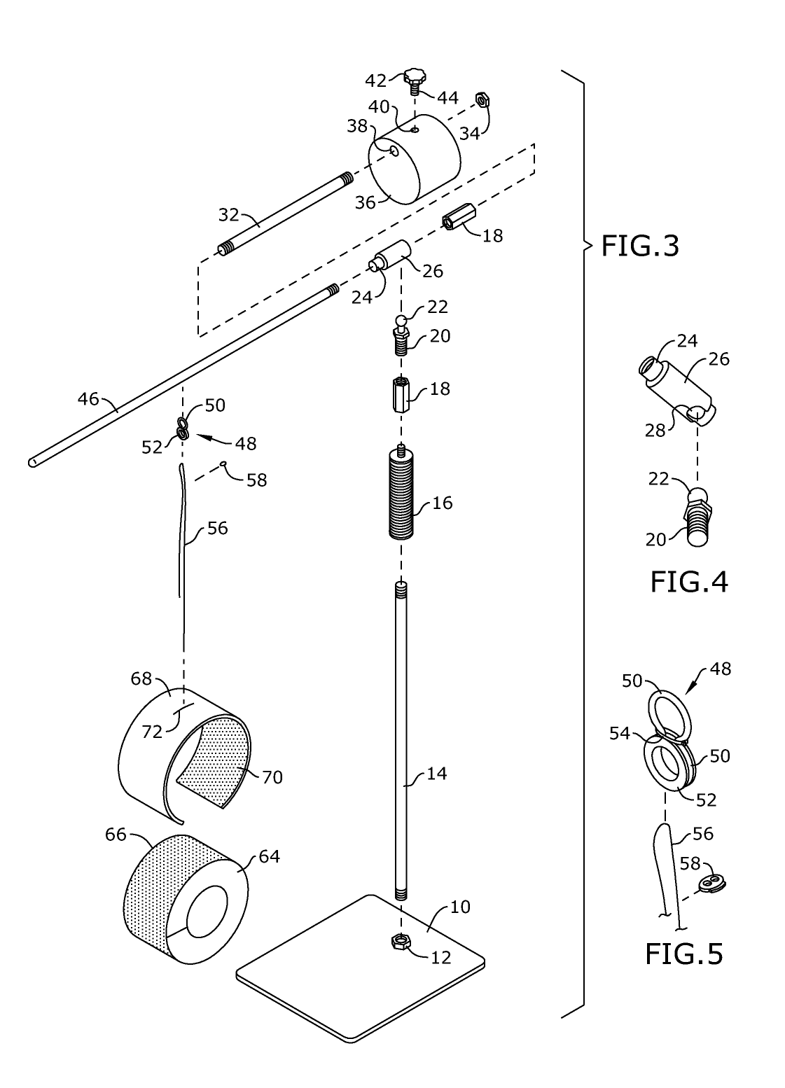

As depicted in FIGS. 1-3, the support apparatus is configured to support a limb of user 74 and permit an uninterrupted range of motion of the limb. It shall be appreciated that the limb can be an arm or leg of user 74. The support apparatus comprises base plate 10, riser bar 14, antenna spring 16, counterweight 36, counterweight bar 32, boom 46, cuff line 56 and cuff fabric covering 64.

Base plate 10 is a rectangular plate having base plate nut 12 welded to the top surface. Riser bar 14 has approximately a ⅜″ diameter with threaded ends. The bottom end of riser bar 14 engages with base plate nut 12. This secures riser bar 14 generally perpendicular to base plate 10. The top end of riser bar 14 is coupled to the bottom end of antenna spring 16. The top end of antenna spring 16 is connected to a joint assembly, which comprises hex coupling 18, ball coupling post 20, ball coupling 22 and socket sleeve 26. Specifically, hex coupling 18 is a ⅜″×1⅛″ member comprising an interior threaded opening having 24 threads per inch (“TPI”). Hex coupling 18 engages with antenna spring 16 and ball coupling post 20. As depicted in FIG. 4, ball coupling 22 is inserted within socket slot 28 of socket sleeve 26.

Socket sleeve 26 is connected to a generally horizontal arm assembly comprising boom 46, counterweight bar 32 and counterweight 36. Boom 46 is a ⅜″ diameter arm made from steel or aluminum with a threaded end that engages with socket body 24 of socket sleeve 26. Hex coupling 18 is secured to the opposing end of socket sleeve 26 and counterweight bar 32. Counter weight bar 32 has a ⅜″ diameter and opposing threaded ends having 16 TPI. Counterweight 36 is a cylindrical member comprising bar hole 38 and knob post hole 40, and is slidably mounted to counterweight bar 32. Bar hole 38 extends through counterweight 36 along the longitudinal axis of the member and is configured to receive counterweight bar 32. Knob post hole 40 is positioned on a top portion of counterweight 36, which permits the hole to intersect bar hole 38 perpendicularly. Once counterweight 36 is slidably adjusted on counterweight bar 32 to a desired position, knob post 44 of knob 42 is inserted into knob post hole 40. Knob 42 is adjusted until knob post 44 contacts counterweight bar 32 within counterweight 36. This secures counterweight 36 to counterweight bar 32 in a stationary position. Counterweight stop 34 is a ⅜″ diameter, 16 TPI threaded end cap that is welded to an end of counterweight bar 32. It shall be appreciated that the components and fasteners described above may be made from any materials known in the field such as steel, aluminum, alternative materials, or the like.

Line mount assembly 48 is secured to boom 46 and a cuff used to enclose a limb of user 74. As depicted in FIG. 5, line mount assembly 48 comprises O-ring 50 coupled to grommet 52 and secured in place by zip tie 54. This creates an upper loop in O-ring 50 that receives boom 46. Cuff line 56 is inserted through grommet 52 to enable two portions of cuff line 56 to be inserted through a pair of openings in cord stop 58. Cord stop 58 can be slidably adjusted relative to cuff line 56 to adjust the hanging length of the line below boom 46. In a preferred embodiment, cuff line 56 is an eighty pound monofilament line. However, alternative types of lines may be used instead.

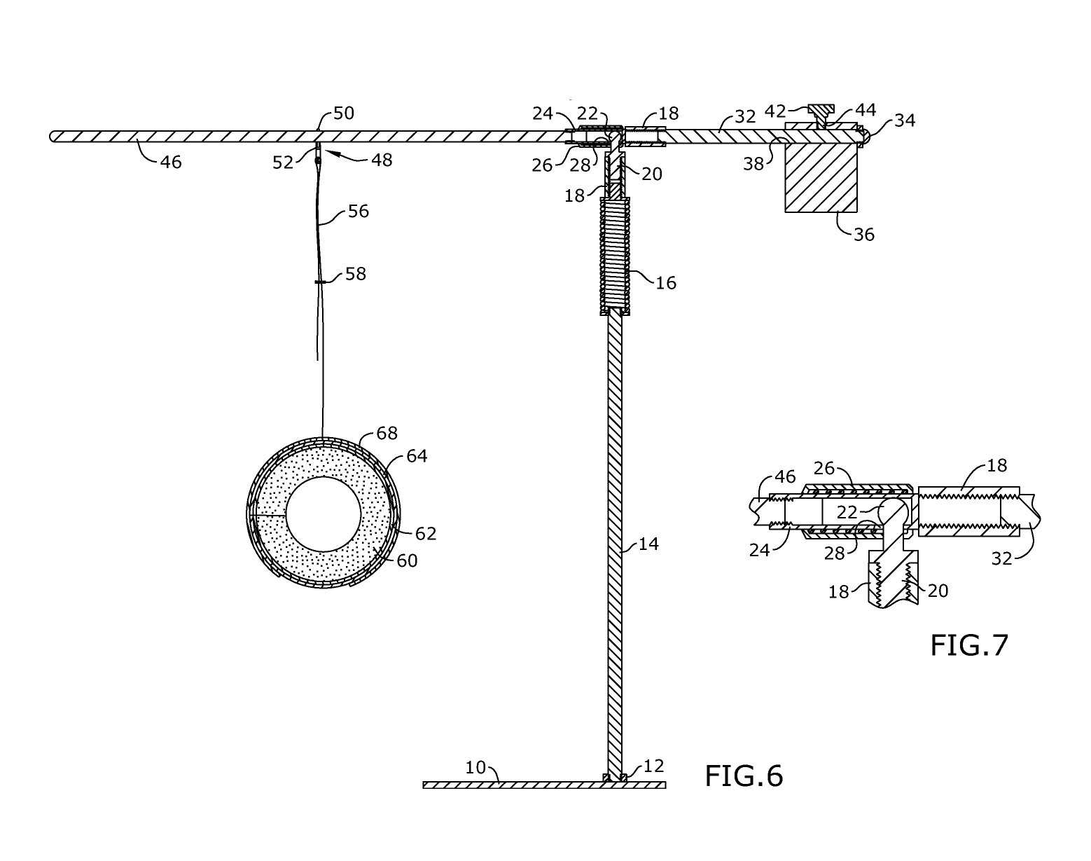

The cuff comprises outer cuff fabric strap 68 and an inner tubular cuff member. Outer cuff fabric strap 68 comprises line slot 72 and hook fasteners 70. The inner tubular cuff member comprises loop fasteners 66, cuff fabric covering 64, cuff flexible plastic layer 62 and cuff foam layer 60. The inner tubular cuff member may have a slot to permit the member to easily be placed around the user's limb. Cuff line 56 is inserted through line slot 72 of outer cuff fabric strap 68. With the user's limb placed within the inner tubular cuff member, outer cuff fabric strap 68 is secured to the inner tubular cuff member by engaging hook fasteners 70 with loop fasteners 66. FIG. 6 depicts a section view demonstrating the layers of the cuff as well as other components of the support apparatus. FIG. 7 depicts a section view of the joint assembly of the support apparatus.

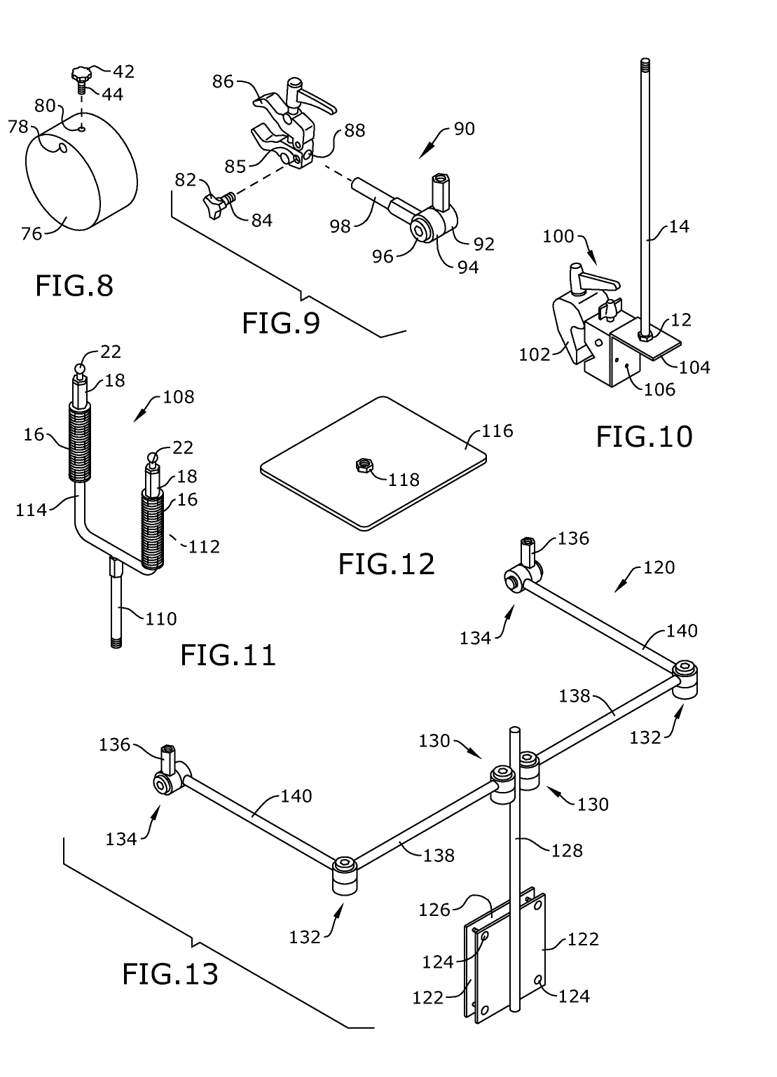

To operate the support apparatus, base plate 10 is placed on a flat surface such as a table, bed, or the like, proximate the limb of user 74 as depicted in FIG. 1. The cuff is secured around the desired limb of user 74. Although the figure depicts the support apparatus being used with an arm, it shall be appreciated that it may be connected to a leg instead. Counterweight 36 is slidably adjusted relative to counterweight bar 32 to adjust the positioning of the generated counterforce, i.e., closer to or away from socket sleeve 26. Once the desired positioning is achieved, counterweight 36 is locked into place via knob 42. In this configuration, the limb of user 74 is fully supported by the cuff connected to boom 46. The counterforce generated by counterweight 36 corresponds to the weight of the limb and permits user 74 to easily move his/her limb. The connection between ball coupling 22 and socket sleeve 26 permits a 360 degree rotation, which provides user 74 with enhanced limb mobility and range of motion. As depicted in FIG. 8, an alternative counterweight 76 comprising bar hole 78 and knob post hole 80 may be used instead. Counterweight 76 comprises a larger diameter than counterweight 36 (4″ compared to 2½″), which may be more desirable in certain applications.

The support apparatus may be secured to alternative structures by using clamp devices and/or a variety of fastening components. As depicted in FIG. 9, elbow assembly 90 is configured for use with bar clamp 86. Bar clamp 86 comprises clamp post hole 85 and clamp bar hole 88. Elbow assembly 90 comprises first hex coupling elbow section 92 pivotably mounted to second hex coupling elbow section 94 by bolt 96. Second hex coupling elbow section 94 is coupled to connector bar 98, which is configured to be inserted within clamp bar hole 88 of bar clamp 86. Once assembled, clamp knob 82 comprising clamp knob post 84 is inserted within clamp post hole 85 to secure bar clamp 86 to elbow assembly 90. As depicted in FIG. 15, bar clamp 86 is coupled to exemplary bar 142, which may be a portion of a structure such as a wheelchair (not shown) that user 74 is located.

FIG. 10 depicts an alternative clamp assembly 100 used to secure the support apparatus to a flat surface such as a board proximate a bed, door, table or alternative structure. Flat surface clamp assembly 100 comprises flat surface clamp 102 coupled to L-bracket 104 by screws 106. Flat surface clamp 102 is secured to the flat surface (not shown) and riser bar 14 is coupled to base plate nut 12 affixed to L-bracket 104. Boom 46, counterweight bar 32, counterweight 36 and line mount assembly 48 can be secured to riser bar 14 as previously described.

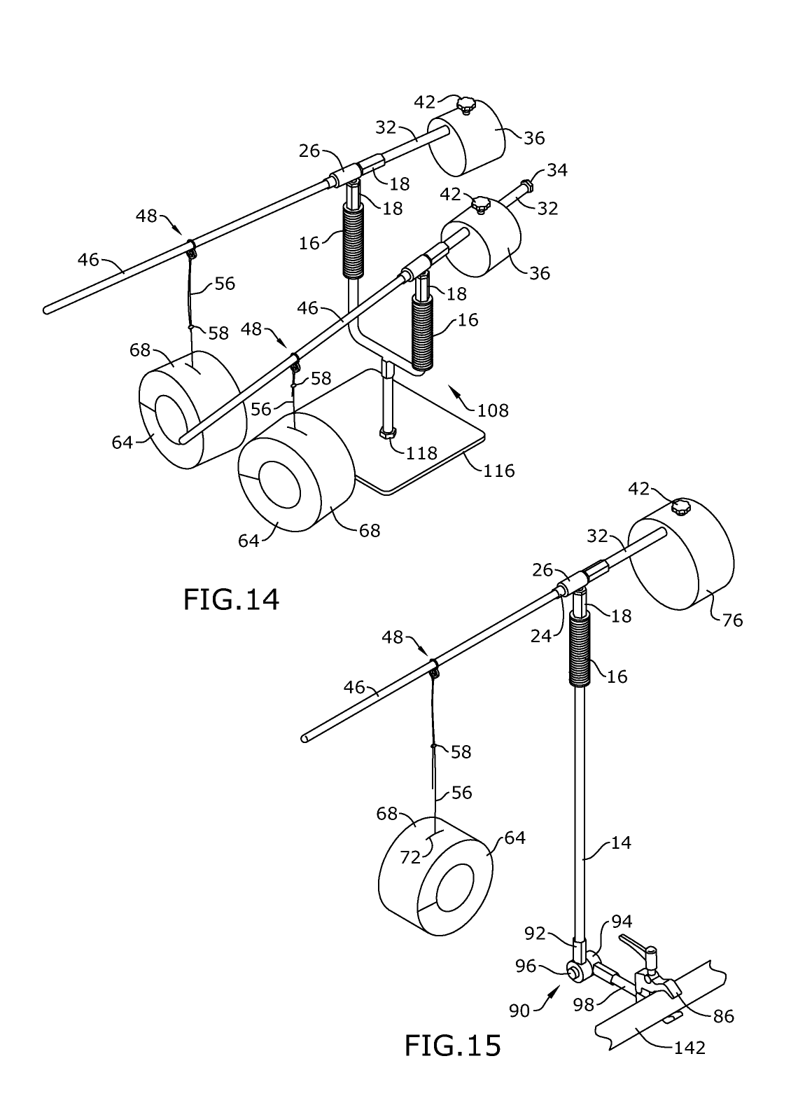

As depicted in FIGS. 11-12, an alternative embodiment of the support apparatus comprises Y-riser assembly 108 comprising Y-riser base bar 110, Y-riser short bar 112, Y-riser long bar 114, antenna springs 16, hex couplings 18 and ball couplings 22. Y-riser short bar 112 and Y-riser long bar 114 are oriented vertically and substantially parallel to each other. Y-riser base bar 110 of Y-riser assembly 108 is secured to alternative base plate 116 via alternative base plate nut 118. As depicted in FIG. 14, booms 46, counterweight bars 32 and counterweights 36 are secured to Y-riser short bar 112 and Y-riser long bar 114 in the same manner as described previously. This configuration permits the support apparatus to be secured to a pair of limbs of user 74 simultaneously, which can be a pair of arms or a pair of legs.

As depicted in FIG. 13, chair mount assembly 120 is shown in an alternative configuration to mount the support apparatus to a chair (not shown). Chair mount assembly 120 comprises mounting plates 122 coupled to center bar 128 coupled to first elbow assemblies 130 coupled to first connector bars 138 coupled to second elbow assemblies 132 coupled to second connector bars 140 coupled to third elbow assemblies 134.

The pair of mounting plates 122 is separated from each other to create chair slot 126. A back portion of the chair (not shown) is inserted through chair slot 126 and mounting plates 122 are tightened around the chair via mounting bolts 124. Each third elbow assembly 134 comprises third elbow hex coupling 136, which is configured to receive a support apparatus (not shown). Therefore, chair mount assembly 120 permits a pair of support apparatuses to be used with a pair of limbs of user 74 when he/she is situated in the chair (not shown).

It shall be appreciated that the components of the support apparatus described in several embodiments herein may comprise any alternative known materials in the field and be of any color, size and/or dimensions. It shall be appreciated that the components of the support apparatus described herein may be manufactured and assembled using any known techniques in the field.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A support apparatus for use with a user to support a limb of the user and permit an enhanced range of motion of the user's limb, the support apparatus configured to adjust a positioning of a counterforce that corresponds to a weight of the limb, the support apparatus comprising:
a generally vertical member comprising a first end coupled to a support structure and a second end comprising a spring mechanism coupled thereto;
a generally horizontal arm assembly rotatably mounted to the spring mechanism by a joint assembly, the horizontal arm assembly comprising a first arm member coupled to a first side of the joint assembly and a second arm member coupled to a second side of the joint assembly;
a limb support assembly coupled to the first arm member of the horizontal arm assembly and comprising a cuff to enclose a portion of the limb; and
a counterweight member slidably mounted to the second arm member, wherein the counterweight member generates the counterforce corresponding to the weight of the limb, thereby permitting the cuff to fully support the user's limb.

2. The support apparatus of claim 1, wherein the counterweight member comprises a cylindrical member comprising a first opening oriented parallel to a longitudinal axis of the cylindrical member and extending therethrough and a second opening oriented perpendicularly to the longitudinal axis of the cylindrical member, wherein the first opening is configured to receive the second arm member of the horizontal arm assembly, wherein the first opening is configured to receive a knob to contact the second arm member within the cylindrical member to secure the counterweight member in a stationary position.

3. The support apparatus of claim 2, wherein the limb support assembly comprises a grommet, an O-ring disposed around the edge of the grommet and secured thereto by a tie to create a loop in the O-ring sufficiently large to receive the first arm member, and a monofilament line disposed through the grommet and coupled to the cuff.

4. The support apparatus of claim 3, wherein the cuff comprises an outer strap coupled to the monofilament line and an inner tubular member detachably coupled to the outer strap.

5. The support apparatus of claim 4, wherein the joint assembly comprises a socket sleeve comprising a first end coupled to the first arm member and a second end coupled to the second arm member, a ball coupling member coupled to a bottom portion of the socket sleeve, and a hex coupling member comprising an upper end coupled to the ball coupling member and a lower end coupled to the spring mechanism.

6. The support apparatus of claim 5, wherein the joint assembly permits a 360 degree rotation of the horizontal arm assembly.

7. The support apparatus of claim 5, wherein the support structure is a base plate.

8. The support apparatus of claim 5, wherein the support structure is a wheelchair.

9. The support apparatus of claim 5, further comprising a clamp coupled to the first end of the vertical member, wherein the clamp is configured to be secured to the support structure.

10. A support apparatus for use with a user to support a plurality of limbs of the user and permit an enhanced range of motion of the user's limbs, the support apparatus configured to adjust a positioning of counterforces that correspond to a weight of each limb, the support apparatus comprising:
a vertical arm assembly comprising a first vertical arm and a second vertical arm, each vertical arm comprising a first end coupled to a support structure and a second end comprising a spring mechanism coupled thereto;
a pair of generally horizontal arm assemblies, each horizontal arm assembly rotatably mounted to the spring mechanism of the first vertical arm or the second vertical arm by a joint assembly, each horizontal arm assembly comprising a first horizontal arm member coupled to a first side of the joint assembly and a second horizontal arm member coupled to a second side of the joint assembly;
a limb support assembly coupled to the first arm member of each horizontal arm assembly and comprising a cuff to enclose a portion of a limb; and
a counterweight member slidably mounted to the second horizontal arm member of each horizontal arm assembly, wherein each counterweight member generates the counterforce corresponding to the weight of the limb, thereby permitting the cuff to fully support the user's limb.
